(19) United States
(12) Patent Application Publication
(10) Pub. No.: US 2026/0041642 A1
(43) Pub. Date: Feb. 12, 2026

(54) Sustained Release Pharmaceutical Composition Comprising Potassium Chloride

(71) Applicant: Granules India Limited, Hyderabad (IN)

(72) Inventors: Krishna Prasad Chigurupati, Hyderabad (IN); Manohar Rao Nitturi, Hyderabad (IN)

(21) Appl. No.: 19/287,941

(22) Filed: Aug. 1, 2025

(30) Foreign Application Priority Data
Aug. 7, 2024 (IN) 202441059625

(51) Int. Cl. A61K 9/20; A61K 9/50; A61K 33/14

(52) U.S. Cl. CPC A61K 9/2081; A61K 9/2009; A61K 9/2013; A61K 9/2027; A61K 9/5042; A61K 33/14

(57) ABSTRACT

The present invention relates to sustained release formulations of Potassium chloride crystals having a mesh size ranging from about 60 to 100 mesh, combined with one or more pharmaceutically acceptable excipients, wherein the potassium chloride crystals are coated with a coating solution comprising cellulosic polymer in an amount up to 9% w/w based on the total weight of the granules and to processes for their preparation.

### FIELD OF THE INVENTION

The present invention relates to sustained release pharmaceutical composition comprising Potassium Chloride crystals, and a process for preparing thereof. More particularly, the present invention relates to sustained release pharmaceutical composition comprising granules formed by coating Potassium chloride crystals with a polymeric solution.

### BACKGROUND OF THE INVENTION

Potassium depletion may occur whenever the rate of potassium loss through renal excretion and/or loss from the gastrointestinal tract exceeds the rate of potassium intake. Such depletion usually develops slowly as a consequence of prolonged therapy with oral diuretics, primary or secondary hyperaldosteronism, diabetic ketoacidosis, severe diarrhea, or inadequate replacement of potassium in patients on prolonged parenteral nutrition. Potassium supplementation is used for patients who have hypokalemia without metabolic alkalosis, in digitalis intoxication and in patients with hypokalemic familial periodic paralysis.

Potassium chloride is conventionally administered in the form of tablets or capsules by the oral route. These dosage forms have the advantages of convenience and ease of administration. It is desired in potassium replacement therapy that the total body potassium level be restored to normal quickly and, for patient convenience, it is desirable that the normal level be sustained for a significant period of time.

One of the leading potassium supplements of Potassium chloride controlled release formulations include K-Tab® available from AbbVie INC, and Klor-Con® available from Upsher-Smith.

U.S. Pat. No. 4,863,743 assigned to Key Pharmaceuticals disclose a controlled release Potassium chloride tablet comprising active ingredient crystals preferably have a mesh size in the range of about 30 to 50 mesh coated with about 9.5 to 18% by weight of a polymeric mixture preferably comprised of from about 9.0 to about 15 parts by weight of ethylcellulose and about 0.5 to about 3.0 parts by weight of hydroxypropylcellulose

U.S. Pat. No. 5,397,574 assigned to Andrx Pharmaceuticals disclose a sustained release solid pharmaceutical composition comprising active ingredient crystals with a mesh size preferably between 20-50 mesh coated with water insoluble coating comprising ethylcellulose of about 70% and 90% by weight of said coating and at least one plasticizer.

U.S. Pat. No. 6,780,437 assigned to Upsher Smith disclose a sustained release solid pharmaceutical composition comprising active ingredient crystals with a mesh size in the range of about 20 to about 60 mesh and a continuous coating on the crystals, the coating consisting of ethylcellulose of about 17% of the total weight of granules.

The present inventors have developed alternative sustained release pharmaceutical compositions of Potassium chloride along with processes for their preparation, that are easier to scale for commercial production and more cost-effective. One such process provides a sustained release composition comprising active ingredient crystals having a smaller particle size. Another such process uses minimum quantity of polymer in the coating thereby providing a sustained release composition which can deliver an effective daily dosage of Potassium chloride.

These compositions exhibited the desired dissolution profile and bioavailability which were also found to be comparable with the marketed formulation.

### SUMMARY OF INVENTION

According to an aspect of the present invention there is provided a sustained release pharmaceutical composition comprising Potassium chloride crystals having a mesh size of about 60 to 100 mesh and one or more pharmaceutically acceptable excipients, wherein the potassium chloride crystals are coated with a coating solution comprising cellulosic polymer in an amount up to 9% w/w based on the total weight of the granules.

In another aspect, the present invention provides Potassium chloride coated crystals combined with one or more pharmaceutically acceptable excipients to prepare a dosage form.

In another aspect, the present invention provides a sustained release tablet composition comprising of Potassium chloride crystals with a mesh size in the range of about 60 to 100 mesh and one or more pharmaceutically acceptable excipients, wherein the potassium chloride crystals are coated with a coating solution comprising cellulosic polymers up to 9% w/w based on the total weight of the granules.

Within the scope of the present invention, it has been discovered that a sustained release composition comprising Potassium chloride crystals with a mesh size in the range of about 60 to 100 mesh coated with cellulosic polymers up to 9% w/w based on the total weight of the granules exhibits an advantageous dissolution profile and/or improved bioavailability. Additionally, this composition ensures high content uniformity and enables efficient production of pharmaceutical dosage forms, both in terms of time and cost.

### DETAILED DESCRIPTION

The term “excipients” as used herein means a component of a pharmaceutical product that is not an active ingredient such as, for example, fillers, diluents, carriers and the like. The excipients that are useful in preparing a pharmaceutical composition are generally safe and nontoxic.

The term “controlled-release” or “sustained release” or “extended release” in accordance with the present invention is intended to provide slow release of the therapeutically active compound during a predetermined period of time from 5 to 10 hours after oral administration of the composition

The term “composition” or “formulation” refers to dosage forms such as tablets, capsules, multiunit particulate systems (MUPS), granules, solid dispersions, pellets, spheres, beads, particles, mini-tablets and the like.

Potassium chloride crystals or API crystals in accordance with the present invention relates to a crystalline form of potassium chloride crystals.

It is to be understood that the singular forms “a”, “an” and “the” include plural referents unless the context clearly dictates otherwise.

As set forth herein, embodiment provides a sustained release Potassium chloride composition and to processes for producing the same. The sustained release compositions provide treatment for potassium deficiency in humans while minimizing adverse side effects.

A further embodiment provides a dosage form comprising the composition or pharmaceutical formulations of the present invention in the form of tablets, beads, granules, powders, sachets, pouches, sprinkles, solutions and suspensions.

In one embodiment, the present invention provides a sustained release pharmaceutical formulations of Potassium chloride comprising active ingredient crystals and one or more pharmaceutically acceptable excipients.

In another embodiment, the formulation provides a sustained release composition comprising Potassium chloride crystals with a mesh size in the range of about 60 to 100 mesh and one or more pharmaceutically acceptable excipients.

The term “mesh size in the range of about 60 to 100” refers to potassium chloride crystals that pass through a 60-mesh sieve but are retained on a 100-mesh sieve.

In another embodiment of the present invention, Potassium chloride crystals with a particle size distribution ranging from about 60 to about 100 mesh are subjected to coating to form polymer coated granules, which are subsequently formulated into a dosage form.

In another embodiment, Potassium chloride crystals with a mesh size in the range of about 60 to 100 mesh are coated with a coating solution comprising cellulosic polymers and one or more pharmaceutically acceptable excipients.

The cellulosic polymers are selected from, but are not limited to, ethylcellulose, methylcellulose, hydroxypropyl cellulose, hydroxypropyl methylcellulose, cellulose acetate, and mixtures thereof.

In another embodiment, the cellulosic polymers are present in an amount of not more than 9% w/w based on the total weight of the granules.

In another embodiment, the cellulosic polymers are present in an amount of up to 9% by weight, i.e., not more than 9%, preferably not more than 8%, and more preferably not more than 7%, based on the total weight of the granules.

In another embodiment, the present invention provides a sustained release tablet composition comprising of Potassium chloride crystals with a mesh size in the range of about 60 to 100 mesh and one or more pharmaceutically acceptable excipients, wherein the potassium chloride crystal are coated with a coating solution comprising cellulosic polymers in an amount up to 9% w/w based on the total weight of the granules.

The present invention offers several significant advantages over conventional potassium chloride formulations. By utilizing active ingredient crystals with a mesh size between approximately 60 and 100, coated with up to 9% by weight of cellulosic polymers, the composition achieves an improved dissolution profile that ensures sustained drug release and enhanced bioavailability. Moreover, the formulation exhibits high content uniformity, guaranteeing consistent dosing accuracy across pharmaceutical units. The use of smaller particle size crystals and minimal polymer coating not only optimizes release kinetics but also enables efficient manufacturing processes, reducing production time and cost. These manufacturing advantages facilitate commercial scalability and make the process economically viable for large-scale pharmaceutical production.

The pharmaceutically acceptable excipients used in the coating solution includes, but not limited to solvents (alcohol, water) plasticizers (e.g. PEG), anti-tack agents (e.g. Talc, colloidal silica), anti-foam agents, colorants, fillers/extenders, flavorants, and surfactants (e.g sodium lauryl sulfate).

The coatings as disclosed herein may be applied according to conventional coating methods comprising cellulosic polymer and one or more pharmaceutically acceptable excipients, from a coating solution that utilizes an alcohol and water solvent.

In another embodiment of the present invention the sustained release composition of the present invention is combined with one or more pharmaceutically excipients to prepare a dosage form.

In a particular embodiment of the present invention, one or more pharmaceutically acceptable excipients as disclosed here in include but not limited to binders, lubricants, anti-adherents, glidants, granulating aids, fillers, disintegrants, antioxidants, stabilizers, preservatives, neutralizing agents, buffering agents, tonicifiers, moisture absorbents, colorants, flavorants, sweeteners, sugars, and taste-masking agents, suspending agents, thickening agents, gelling agents, solvents, solubilizers, surfactants, absorption enhancers, emulsifying agents, and combinations thereof.

Examples of these pharmaceutically acceptable excipients in the component of the present invention include, but are not limited to, binders/fillers: microcrystalline cellulose, silicified microcrystalline cellulose, polyvinylpyrrolidone, hydroxypropyl cellulose, starch, pregelatinized starch, starch paste, lactose, mannitol, sorbitol, xylitol, sucrose, calcium phosphate, calcium carbonate, ethylcellulose, methylcellulose, and Acacia; lubricants/anti-adherents/glidants/granulating aids: talc, sodium lauryl fumarate, fumed silicon dioxide, colloidal silica, titanium dioxide, kaolin, magnesium stearate, calcium stearate, stearic acid, hydrogenated vegetable oils, and sodium lauryl sulfate; disintegrants: sodium starch glycolate, croscarmellose sodium, cross-linked polyvinylpyrrolidone, and alginic acid; antioxidants/stabilizers/preservatives: riboflavin, tocopherol, vitamin E TPGS, BHT, BHA, cysteine and derivatives, ascorbates, sorbates, benzoates, propionates, bicarbonates, thiosulfates, metabisulfites, EDTA, carrageen, gums and benzyl alcohol; neutralizing agents: acids such as malic acid, citric acid, tartaric acid, ascorbic acid, oleic acid, capric acid, caprylic acid, benzoic acid, polyacids, acidic ionic resins, and other acidic excipients; suspending agents/thickening agents/gelling agents: mineral oils, vegetable oils, silicon dioxide, various gums such as xanthan gum, locust bean gum, gum Arabic, alginates, carbopols, polyvinyl alcohols, carrageenan, gelatin, starches; or mixtures thereof.

In an embodiment of the present invention provides process for preparing a pharmaceutical composition of Potassium chloride wherein the process comprises the steps of coating the Potassium chloride crystals with a mesh size in the range of about 60 to 100 mesh with cellulosic polymer solution in an amount up to 9% by weight of the total weight of granules and mixed with one or more pharmaceutically acceptable excipients to produce the dosage form.

In another embodiment of the present invention provides process for preparing a solid dosage form of Potassium chloride, wherein the process comprises the steps of coating the Potassium chloride crystals with a mesh size in the range of about 60 to 100 mesh with cellulosic polymer solution in an amount up to 9% by weight of the total weight of granules and mixed with one or more pharmaceutically acceptable excipients to produce a uniform blend and finally compressing the blend into tablets or filled into capsules.

In another embodiment of the present invention provides process for preparing a solid dosage form of Potassium chloride, wherein the process comprises the steps of coating the Potassium chloride crystals with a mesh size in the range of about 60 to 100 mesh with cellulosic polymer solution in an amount up to 9% by weight of the total weight of granules and wherein the process time cellulosic polymer coating is less than 8 hours, preferably less than 6 hours.

In another embodiment one or more seal coats of different coating materials are provided as over coat on the tablet dosage form.

The overcoat can be a moisture barrier coat, a protection coat, a seal coat, a taste-masking coat, a flavor coat, a polish coat, a color coat, or any other cosmetic coats. Suitable coating materials for such an overcoat are known in the art, including, but are not limited to, cellulosic polymers such as hydroxypropyl methylcellulose, hydroxypropylcellulose, microcrystalline cellulose carrageenan, ethylcellulose and one or more pharmaceutically acceptable excipients.

### EXAMPLES

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### Example—1: Ethylcellulose+Hydroxypropyl Cellulose Coated Granules of Potassium chloride

| S. NO | Ingredients | % w/w |
|---|---|---|
| 1 | Potassium Chloride | 91.0 |
| 2 | Ethylcellulose | 8.7 |
| | (Ashland EC | |
| | N100/20/10 Pharm Ultra) | |
| 3 | Hydroxypropyl | 0.2 |
| | Cellulose | |
| | (Klucel LF) | |
| 4 | Methanol | q. s. |
| 5 | Purified Water | q. s. |
| | Total | 100 |
| | Percentage weight | 9.89% |
| | gain (%) | |
| | Process time | about 2 hrs |

### Brief Manufacturing Process:

Ethylcellulose coating solution was formed by dissolving ethylcellulose (Ashland EC N100/20/10 Pharm Ultra sourced from Ashland) in solvent mixture of methanol and purified water followed by addition of hydroxypropyl cellulose (Klucel LF sourced from Ashland). The formed coating solution was sprayed onto the Potassium chloride crystals passed through 60 mesh and retained on 100 mesh in a bottom spray coater apparatus to form coated granules of Potassium chloride.

### Example—2: Ethylcellulose Coated Granules of Potassium Chloride

| | | Ex-2a | Ex-2b |
|---|---|---|---|
| S. NO | Ingredients | % w/w | % w/w |
| 1 | Potassium Chloride | 95.0 | 99.0 |
| 2 | Ethylcellulose | 5.00 | 1.00 |
| | (Ashland EC | | |
| | N100/20/10 Pharm Ultra) | | |
| 3 | Methanol | q. s. | q. s. |
| 4 | Purified Water | q. s. | q. s. |
| | Total | 100 | 100 |
| | Percentage weight | ~5.2 | ~1.0 |
| | gain (%) | | |
| | Process time | about 2 hrs | about 1 hr |

### Brief Manufacturing Process:

Ethylcellulose coating solution was formed by dissolving ethylcellulose (Ashland EC N100/20/10 Pharm Ultra sourced from Ashland) in solvent mixture of methanol and purified water. This coating solution was sprayed onto the Potassium chloride crystals passed through 60 mesh and retained on 100 mesh in a bottom spray coater apparatus to form coated granules of Potassium chloride.

### Example—3 Compressed Dosage Forms of Potassium Chloride Tablets

| | | Ex-3a | Ex-3b | Ex-3c |
|---|---|---|---|---|
| S. NO | Ingredients | % w/w | % w/w | % w/w |
| 1 | Potassium Chloride | 86.0 | 75.0 | 70.0 |
| 2 | Ethylcellulose | 2.0 | 6.5 | 6.0 |
| | (Ashland EC | | | |
| | N100/20/10 Pharm Ultra) | | | |
| 3 | Methanol | q. s. | q. s. | q. s. |
| 4 | Purified Water | q. s. | q. s. | q. s. |
| 5 | Hydrogenated Vegetable | 2.5 | 3.5 | 5.0 |
| | oil, TYPE 1, NF | | | |
| | ((Lubritab ®) | | | |
| 6 | Stearic acid | 2.5 | 3.5 | 5.0 |
| 7 | Silicon dioxide | 1.5 | 2.5 | 4.0 |
| 8 | Polyvinylpyrrolidone | 2.5 | 3.0 | 4.0 |
| | (Pvp K-30) | | | |
| 9 | Talc | 1.0 | 1.5 | 2.0 |
| 10 | Film coating material | 2.0 | 3.0 | 4.0 |
| | Total % | 100.0 | 100.0 | 100.0 |
| | Percentage | ~2.3 | ~8.6 | ~8.5 |
| | weight gain | | | |
| | Process time | about | about | about |
| | (in hrs) | 1 hr | 1.5 hr | 2 hrs |

### Brief Manufacturing Process:

Ethylcellulose coating solution was formed by dissolving ethylcellulose (Ashland EC N100/20/10 Pharm Ultra sourced from Ashland) in solvent mixture of methanol and purified water and the formed coating solution was sprayed onto the Potassium chloride crystals passed through 60 mesh and retained on 100 mesh in a bottom spray coater apparatus to form coated granules of Potassium chloride. The coated particles were blended with hydrogenated vegetable oil (Lubritab® sourced from JRS Pharma), Stearic acid, Silicon dioxide, Polyvinylpyrrolidone (Pvp K-30 sourced from Ashland) and talc followed by compression. The compressed tablets were coated with film coating dispersion prepared by dispersing the film coating materials in water under continuous stirring and finally the tablets were cured for 1-3 hrs at a temperature of about 50° C.-70° C.

While the foregoing pages provide a detailed description of the preferred embodiments of the invention, it is to be understood that the summary, description and examples are illustrative only of the core of the invention and non-limiting. Furthermore, as many changes can be made to the invention without departing from the scope of the invention, it is intended that all material contained herein be interpreted as illustrative of the invention and not in a limiting sense.

We claim:

1. A sustained release pharmaceutical composition comprising Potassium chloride crystals having a mesh size of about 60 to 100 mesh and one or more pharmaceutically acceptable excipients, wherein the potassium chloride crystals are coated with a coating solution comprising cellulosic polymer in an amount up to 9% w/w based on the total weight of the granules.

2. The sustained release composition as claimed in claim 1, where in the potassium chloride crystal having a mesh size of about 60 to 100 mesh refers to crystals that pass through 60 mesh and are retained on a 100 mesh.

3. The sustained release composition as claimed in claim 1, wherein the cellulosic polymers are selected from ethylcellulose, methylcellulose, hydroxypropyl cellulose, hydroxypropyl methylcellulose, cellulose acetate, and mixtures thereof.

4. The sustained release composition as claimed in claim 1, wherein the cellulosic polymer is present in an amount of not more than 9% w/w based on the total weight of the granules.

5. A sustained release tablet composition comprising Potassium chloride crystals having a mesh size of about 60 to 100 mesh and one or more pharmaceutically acceptable excipients, wherein the potassium chloride crystals are coated with a coating solution comprising cellulosic polymer in an amount up to 9% w/w based on the total weight of the granules.
